(19) United States
(12) Patent Application Publication
(10) Pub. No.: US 2004/0010170 A1
(43) Pub. Date: Jan. 15, 2004

(54) Para-xylene and ethylbenzene separation from mixed C8 aromatics

(76) Inventor: George H. Vickers, Aurora, IL (US)

Correspondence Address: BP America Inc., Docket Clerk, Law Department, M.C. 2207A, 200 East Randolph Drive, Chicago, IL, 60601-7125, US

(21) Appl. No.: 10/316,619

(22) Filed: Dec. 11, 2002

Related U.S. Application Data
(60) Provisional application No. 60/347,063, filed on Jan. 9, 2002.

(51) Int. Cl. C07C 6/00; C07C 5/00

(52) U.S. Cl. 585/319

(57) ABSTRACT

A para-xylene recovery process including adsorption apparatus containing a para-selective adsorbent to separate para-xylene and ethylbenzene from a C₈ aromatic hydrocarbon stream, said process operated isothermally in the vapor phase at elevated temperatures and pressures, and continuously controlled in real-time by analysis of an effluent stream using NIR spectroscopy.

### CROSS REFERENCE TO RELATED APPLICATIONS

This application claims benefit under 35 USC 119(e) of U.S. Provisional Application No. 60/347,063, filed Jan. 9, 2002, incorporated by reference herein.

### BACKGROUND OF THE INVENTION

The present invention relates to a method for controlling and optimizing a para-xylene separation process using near-infrared (NIR) spectroscopy. More particularly, the present invention relates to a method and an apparatus for controlling and optimizing a para-xylene separation and isomerization process wherein the components of the process stream are measured using NIR spectroscopy in an in-line or on-line manner, providing real-time data for process control and optimization.

In the petrochemical production chain, one of the most important streams is the C₆ to C₈ aromatics stream containing benzene, toluene, and xylenes (BTX), which is a source of raw materials for high value downstream products. Of the C₈ aromatics, para-xylene (pX), a raw material in the manufacture of terephthalic acid, is the most desirable.

Because the boiling points of ethylbenzene (EB), ortho-xylene (oX), meta-xylene (mX) and para-xylene (collectively referred to as “C₈ aromatics”) are close, they are difficult to separate by fractional distillation. Alternative methods for isolating pX from the C₈ aromatics stream are well known in the art, including fractional crystallization and liquid-phase adsorption. Processes in wide commercial use for the production of pX have typically included combinations of isomerization with fractional crystallization.

Fractional crystallization is a separation process that takes advantage of the fact that pX crystallizes at a higher temperature than the other isomers. The crystallization temperature of pX is 13.3° C.; oX crystallizes at −25.2° C.; and mX crystallizes at −47.9° C. The three isomers also form two binary eutectics of importance, the pX/mX and the pX/oX. Depending on the starting composition of the mixture, as pX crystallizes from the mixture, the mother liquor approaches these binary eutectic compositions. When one of these binary eutectics is reached, both isomers will then co-crystallize, lowering the pX purity. In commercial practice, pX is crystallized so that the binary eutectics are only approached but not reached. Typically, the equilibrium concentration of pX in a mixed C₈ aromatic stream from a catalyst reactor is about 22 wt %. Avoiding the eutectic point of this mixture limits the pX removed per pass to about 65% of that amount.

A significant reduction in the amount of meta-xylene and ortho-xylene in the C₈ aromatics stream sent to a crystallization section would increase capacity and decrease operating costs. Enriching the para-xylene content of a stream going to the crystallization section may also make it possible to eliminate a crystallizer, saving equipment costs and reducing the amount of energy needed to conduct the crystallization and purification of para-xylene.

Adsorption processes for separating the para-xylene component of a C₈ aromatics stream are widely known and well described in the art. Such processes and adsorbents suitable for use in such processes are described, for example, in U.S. Pat. Nos. 5,329,060, 5,922,924, 3,724,170 and 3,729,523, in British Patent 1,420,796, and in published Chinese Patent Application 1136559.

More recently, in U.S. patent application Ser. No. 09/902,198, filed Jul. 10, 2001, there is disclosed an adsorption process for rapid separation of the desired components from a feed stream containing C₈ aromatics. The process uses selective adsorption (adsorption of the smaller C₈ isomers) and selective desorption (i.e., no isomerization upon desorption) at substantially isothermal temperatures to provide a product stream having enhanced levels of para-xylene and ethylbenzene and a product stream having enhanced levels of ortho-xylene and meta-xylene. The components in these streams can be further separated, for example, by fractional crystallization, to provide substantially pure para-xylene, ethylbenzene, ortho-xylene, and meta-xylene products.

The process is conducted using a non-acidic, molecular sieve-containing adsorbent and at an elevated temperature and pressure to accomplish a rapid adsorption and desorption of the desired components. A non-acidic molecular sieve, such as silicalite (MFI structure type with little to no aluminum), is used to selectively adsorb pX and EB. Desorption from the adsorbent is significantly faster, and reactions of the adsorbed molecules (pX and EB) do not occur. In addition, olefins do not adsorb on the silicalite, so the adsorption capacity of the adsorbent remains high and frequent reconditioning is not required.

Pressure swing adsorption processes are necessarily cyclic processes, and for reasonably sized adsorbent beds the cycle times will be short (seconds to minutes). The cycle timing, flow rates, temperatures and pressures are typically arbitrary, based on prior performance of the adsorbent bed and derived empirically from historic analytical data relating to average effluent composition. The requisite data may be obtained by a variety of well-known analytical methods such as, for example, both on-line and off-line gas chromatography. Gas chromatography requires analysis times of from 5 minutes to about one hour. Where a plurality of streams are to be monitored on-line, the use of a plurality of on-line gas chromatography devices corresponding to the respective sites is advised to avoid delays from sequentially analyzing more than one stream on a single device. For these reasons, on-line gas chromatography has been used only to continuously check a few essential components and streams associated with an optimum process operation. Obtaining samples for measurement of required components in the laboratory, using off-line gas chromatography, requires time additional to the already lengthy analysis time. Gas chromatography thus is not well suited for effective real-time control of processes with short cycle times.

Spectroscopic methods have also been disclosed in the art for this purpose. In WO 99/02973, there are disclosed NIR methods for analysis of a variety of hydrocarbon streams. In U.S. Pat. No. 6,162,644, samples of a hydrocarbon process stream are collected, cooled, condensed and isolated, and then analyzed using NIR. According to patentees, the online process monitoring may be accomplished within 15 minutes.

The use of NIR or FT-IR spectroscopy to control a xylene separation process is disclosed in U.S. Pat. No. 5,470,482. According to patentees, the disclosed techniques require no sample preparation, are accurate, rapid and non-destructive, and can be performed on-line or in-line to provide data in less than a minute, which translates into the ability to immediately and precisely control the value of a characteristic.

Optimizing cycle timing in an adsorption separation process—such as by cutting off feed flow to end the adsorption step before breakthrough of adsorbate—requires real-time information about the feed and effluent streams. Otherwise, cutoff of the feed to end the adsorption step may actually occur after breakthrough or well before, significantly lowering efficiency. Means for rapidly and continuously analyzing feed and product streams under operating conditions in real-time would allow continuous control and optimization of the cycle timing, flow rates and pressures, improving the overall process efficiency.

### SUMMARY OF THE INVENTION

In one embodiment, the present invention is an apparatus for directly identifying and quantifying components in a process stream from a para-xylene production unit, preferably including an adsorption process for separation of xylene isomers from a C₈ aromatic hydrocarbon feed stream, said apparatus comprising a microprocessor and a near-infrared (NIR) spectrometer including a sample cell through which at least a portion of a process stream may flow, a source to emit NIR radiation or light and pass the NIR light through said process stream, a detector to detect transmitted NIR light at selected wavelengths and to generate absorbance data due to absorbance of the NIR light by the components of said process stream, wherein each of the components absorbs NIR light at one or more of the NIR wavelengths, and an output apparatus to provide the absorbance data to the microprocessor, wherein the microprocessor is programmed to identify and quantify each of the plurality of said components based upon the absorbance data and calibration data.

In one embodiment, the present invention is a method for identifying and quantifying components in a process stream from a para-xylene production process, comprising (a) advancing a portion of the process stream through a sample cell coupled to a NIR spectrometer; (b) scanning the portion in the sample cell with NIR light at a plurality of NIR wavelengths, wherein each of the components of said process stream absorbs NIR light at one or more of the plurality of NIR wavelengths; (c) detecting the NIR light passing through the sample cell and generating absorbance data for each of the components; and (d) quantifying each of the components by applying the absorbance data to a calibration equation for each component in a microprocessor programmed to quantify each of the components.

In one embodiment, the invention is a method for controlling the operation of an adsorption process based on the real-time quantitative analysis of components in a process stream from a para-xylene production process, comprising (a) advancing a portion of the process stream through a sample cell coupled to a NIR spectrometer; (b) scanning the portion in the sample cell with NIR light at a plurality of NIR wavelengths, wherein each of the components of said process stream absorbs NIR light at one or more of the plurality of NIR wavelengths; (c) detecting the NIR light passing through the sample cell and generating real-time absorbance data for each of the components; (d) calculating, in a microprocessor programmed therefor, real-time quantitative data for the effluent from the calibration equation and the real-time absorbance data; and (e) controlling process conditions to optimize production of at least one of the components based on the real-time quantitative data.

The adsorption process will be operated to produce a hydrocarbon product stream enriched in pX content relative to the feed stream. In one embodiment, the adsorption process is operated so as to produce substantially pure pX. In one embodiment, the adsorption process is operated so as to produce a mixture of pX and EB substantially free of mX and oX. In one embodiment, the adsorption process is operated so as to produce a mixture comprising pX and EB which contains no more than a total of about 50 mole percent of mX and oX.

The adsorption process may be further operated to produce a hydrocarbon stream enriched in mX and oX content relative to the feed stream In one embodiment, the adsorption process may be operated so as to produce a hydrocarbon stream comprising a mixture of mX and oX substantially free of pX and EB. In one embodiment, the process may be operated so as to produce a hydrocarbon stream comprising a mixture of mX and oX which contains no more than a total of about 25 mole percent of pX and EB.

The present invention provides the real-time information needed to allow improved control and immediate, continuing optimization of an adsorption process during the operation for which the information is obtained, thus providing real-time analyses and process control. While the following description is directed to the separation of components of a C₈ stream by an adsorption process, it is to be understood that this is for illustrative purposes only, and that the invention, applicable broadly to hydrocarbon process streams generally, is not so limited.

### BRIEF DESCRIPTION OF THE DRAWINGS

FIG. 1 is a schematic representation of an adsorption unit vessel together with an NIR spectrometer analysis apparatus in accord with an embodiment of the invention, showing the adsorption vessel, feed lines, NIR sample cells and microprocessor, NIR spectrometer and optical multiplexer with fiber optic connections.

FIG. 2 is a schematic representing the stages of an adsorption cycle for pX/EB separation which operates at substantially constant system pressure and uses an inert sweep gas such as, for example, CH₄, CO₂, H₂, N₂, or He, to accomplish desorption.

FIG. 3 is a graphical presentation of the total hydrocarbon partial pressure and the pX concentration of the hydrocarbon phase in the adsorption effluent from the adsorption vessels for two cycles from the run of Example 1.

FIG. 4 is a graphical presentation of the total hydrocarbon partial pressure and the pX concentration of the hydrocarbon phase in the adsorption effluent from the adsorption vessels for two cycles from the run of Example 2.

FIG. 5 is a graphical presentation of the estimated amounts of the individual C₈ aromatic components in the adsorption effluent from the adsorption vessels for two cycles from the run of Example 2.

FIG. 6 is a graphical presentation of the total hydrocarbon partial pressure and the pX concentration of the hydrocarbon phase in the desorption effluent from the adsorption vessels for 14 cycles of the run of Example 3, shown in two cycle overlay.

FIG. 7 is a graphical presentation of the total hydrocarbon partial pressure and the estimated amounts of the individual C₈ aromatic components in the desorption effluent from the adsorption vessels for two cycles from the run of Example 4, showing estimated amounts of the components.

FIG. 8 is a graphical presentation of the total hydrocarbon partial pressure and the estimated amounts of the individual C₈ aromatic components in the desorption effluent from the adsorption vessels for 10 cycles of the run of Example 4, shown in two cycle overlay.

### DETAILED DESCRIPTION

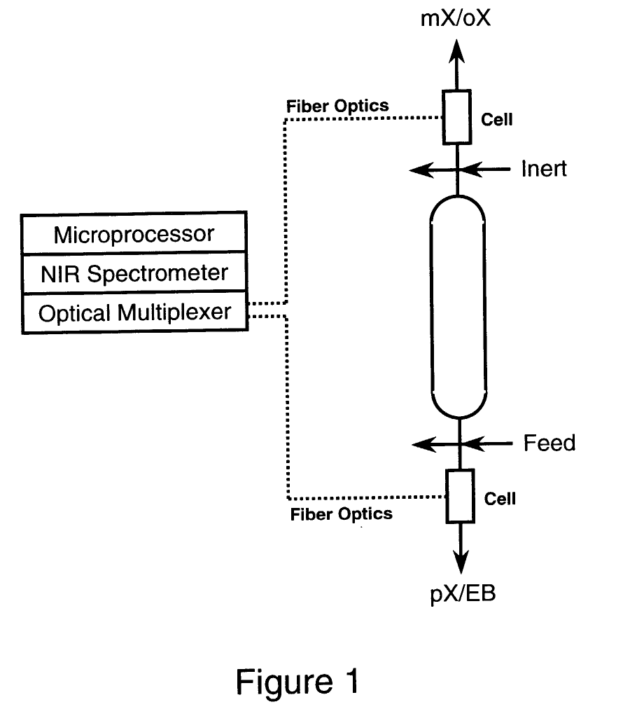

In one embodiment, the invention comprises a near-infrared (NIR) analyzer system adapted to monitor and analyze a process stream of an adsorption unit for separation of pX and EB from mX and oX, substantially as shown schematically in FIG. 1. The adsorption unit is a component suitable for use with a para-xylene production unit, for example, a para-xylene production unit comprising a catalyst reactor for isomerization of aromatics. Such para-xylene production units typically further comprise a catalyst reactor for ethylbenzene conversion, one or more distillation columns for separation of aromatics and, optionally, a fractional crystallization unit for separating para-xylene from ethylbenzene. Typically, such para-xylene production units include a plurality of such adsorption units, operating in parallel.

The NIR analyzer system may comprise a near-infrared (NIR) spectrometer, a fiber optic multiplexer, at least one sample cell, an optical attenuator and a detector sensitive to NIR light.

The sample cell will be placed in the adsorption process unit and connected to an inlet or outlet port thereof in a manner such that at least a portion of the process stream will pass through the cell under the prevailing operating conditions.

Optical connections between the sample cell and the system components, including the spectrometer, multiplexer, attenuator and detector may be made using suitable fiber optic cables such as, for example, 300-micron diameter core, low-OH silica fiber optic cables. Optically coupling the sample cell in this manner allows the cell to be operated within the harsh environment of process hardware to thereby minimize sampling time. The spectrometer and other sensitive electronic components in optical communication with the cell may be located remotely, in a control room or other suitable location. Other suitable NIR analyzers may be located in closer proximity to the sample cell and may even be directly connected to the sample cell without the use of fiber optics. The entire NIR analyzer system is under computer control using process-monitoring software.

The spectrometer may be selected from the wide variety of NIR spectrometers well known and widely used in the art for analysis purposes, including, for example, Fourier-transform near-infrared (FT-NIR) spectrometers, diode array NIR spectrometers, scanning diffraction grating NIR spectrometers, acousto-optic tunable filters (AOTF), filter photometers and the like.

The analyzer system employed in the following description included an FT-NIR spectrometer; other spectrometer types may be similarly adapted for use in the practice of this invention.

The NIR light, modulated by the spectrometer, is delivered to the multiplexer by a fiber optic cable, directed to either the sample cell or the attenuator through additional fiber optic cables, and then delivered through another fiber optic cable. The light passes through either the sample cell or the attenuator, is returned to the multiplexer and then delivered through additional fiber optic cables to the Indium-Gallium-Arsenide (InGaAs) detector, where the light intensity is converted into an electrical signal that is processed into an intensity spectrum. The attenuator channel provides a reference, or background spectrum, to the NIR analyzer; the sample cell channel provides a foreground spectrum. The negative base-10 logarithm for the ratio of the foreground over the background spectrum is the absorbance spectrum of the sample.

Alternatively, the background spectrum may be measured through the sample cell, making the attenuator unnecessary. This would require that the sample cell be filled with a non-absorbing material such as nitrogen, hydrogen or the like on a periodic basis to measure the background spectrum.

Where a plurality of process streams are to be analyzed, each may be provided with separate sample cells and accessed by the NIR analyzer as different channels on the multiplexer. At least a portion of entire stream flow will be passed through the cell, thereby keeping the lag time in the response from the NIR analyzer to a minimum.

Spectra are conveniently measured by averaging 16 scans at a resolution of 16 cm⁻¹ over a wavenumber range from 9000 to 4500 cm⁻¹. Fewer numbers of scans, for example, 8 or as few as 2, may be used, if desired, to shorten the measurement time. The low resolution of 16 cm⁻¹ also shortens the measurement time, yet retains the necessary information content of the spectra to perform real-time analysis.

The sample cell of the NIR analyzer system will be constructed to operate at the elevated temperatures and over the range of operating pressures employed in the process equipment, generally from less than 50 psia to more than 400 psia. The temperature of the cell will be maintained at greater than 250° C., and preferably greater than 300° C. to assure that the streams will be entirely in the gas phase at any pressure within the operating range, so that nothing condenses on the sample cell windows that could obscure the path of light.

The sample cell path length will be determined in part by the absolute concentration range of the components of the process stream. Conveniently, the sample cell may have a path length of 10 cm from the input to output window, providing a spectral absorbance peak above the baseline of about 0.01 times the hydrocarbon partial pressure in psia, when intended for use from about 20 to 100 psia of hydrocarbon partial pressure. The hydrocarbon partial pressures may vary from near zero to 100% of the absolute pressure. For higher or lower partial pressures, a shorter or longer path length, respectively, would be preferred.

The NIR region of the electromagnetic spectrum falls between the visible region (which ends at roughly 14000 cm−1) and the infrared region (which begins at about 4000 cm−1). This region of the spectrum involves relatively weak, broad absorption bands due to overtones and combination bands arising from the vibration of CH, NH, OH, and SH bonds in molecules. The wavenumber range of 9000 to 4500 cm⁻¹ includes the first overtone of the C—H absorbance bands and a portion of the first combination bands. This region of frequencies also contains the second overtone and combination bands, which might be useful under different process conditions (i.e. higher pressure) and/or analyzer configurations (i.e. longer path length).

Particular bands have been disclosed in the art for use in the determination of aromatics, including:

For benzene: 4762-4630 cm⁻¹; 6250-5988 cm^{31 1}; and 5618-5382 cm^{-1;}.

For xylenes: 7576-6536 cm⁻¹; 5000-4902 cm⁻¹; 6250-5988 cm⁻¹; 8929-8237 cm⁻¹; 5618-5382 cm⁻¹; 4762-4630 cm⁻¹; 10638-10101 cm⁻¹; 11765-10753 cm⁻¹; and 9852-9569 cm⁻¹.

For alkylbenzenes: 8651-7911 cm⁻¹; 6250-5988 cm⁻¹; 8237-8130 cm⁻¹; 7576-6757 cm⁻¹; 11765-11111 cm⁻¹; 9852- 9569 cm⁻¹; 8237-7911 cm⁻¹; and 5076-4902 cm⁻¹.

Those skilled in the art will recognize that still further bands and specific frequencies within the NIR range may also be found suitable for the purposes of this invention.

Several types of determinations may be made by the NIR analyzer system to monitor the operation of the process without fully quantifying the components of the process stream. For example, the hydrocarbon partial pressure may be conveniently estimated from the difference between the absorbance values at 5893.4 and 6495.0 cm⁻¹, where 5893.4 cm⁻¹ corresponds to an aromatic C—H absorbance band and 6495.0 cm⁻¹ is a point on the baseline with no hydrocarbon absorbance. The hydrocarbon partial pressure will be approximately 100 times this difference at the typical sample cell operating temperature range of 300 to 350° C.

The NIR analyzer system may also be used to provide a semi-quantitative indication of the para-xylene (pX) concentration in the hydrocarbon phase that shows approximate increases and decreases over time. This calculation will be performed using the following formula:

pX (wt %)=m*(A(5847.1)+A(5908.8)−A(5978.2)−A(5785.4))/(A(5893.4)−A(6495.0))+b

where A(xxxx.x) is the measured absorbance at xxxx.x cm⁻¹, while m and b are calibration coefficients that convert the absorbance term into a concentration.

Absorbance values at 5847.1 and 5908.8 cm⁻¹ are directly correlated to pX, while absorbance values at 5978.2 and 5785.4 cm⁻¹ are inversely correlated to pX. Dividing by the absorbance difference between 5893.4 cm⁻¹ and 6495.0 cm⁻¹ compensates for the hydrocarbon partial pressure to produce a value that tracks the pX content in the hydrocarbon phase.

Spectral determinations are rapid; for most commercially-available FT-NIR instruments, the spectral measurement time is only about 5 seconds to average 16 scans at a resolution of 16 cm⁻¹ over a wavenumber range from 9000 to 4500 cm⁻¹, and other instruments may be found to require less time. The calculation times will be a function of the microprocessor speed.

A complete quantitative determination of the ortho-xylene (oX), meta-xylene (mX), para-xylene (pX), and ethyl-benzene (EB) components of a hydrocarbon stream may be made using correlation models developed with various regression methods well known to those skilled in the art such as, for example, partial-least squares (PLS) regression. Preliminary PLS models may be developed using spectra generated by mathematically combining pure component spectra of oX, mX, pX and EB, as well as of other hydrocarbons as required, obtained by vaporizing these compounds and flowing them through the sample cell.

The spectra of the pure components will be measured at a constant temperature and known pressure. Because the measured absorbance values are linearly related to concentration, and the low densities in the gas phase limit molecular interactions, mathematically produced spectra from these pure component spectra will be nearly identical to those that would be measured for actual mixtures. Accordingly, they provide a very simple, yet effective, calibration set for the preliminary PLS models. To account for the expected absolute and partial pressure variations within the process hardware, and possible fouling of the sample cell, the calibration spectra will be pre-processed prior to being used to develop the models, conducted according to standard methods well known to those skilled in the art and widely used for these purposes. Subsequent spectra of process streams measured in real-time during the operation of the process will be processed in an identical fashion prior to the quantitative determinations using the preliminary PLS models.

The accuracy of these preliminary PLS models can be further improved using spectra of mixtures containing the appropriate hydrocarbons. These spectra can be used to supplement the mathematically produced spectra or entirely on their own to produce more accurate PLS models. Alternately, the relationship between the true compositions of these mixtures and the corresponding results from the preliminary PLS models can be used to accurately adjust any future results determined using the preliminary PLS models. All of these techniques are well known to those skilled in the art and are widely used for these purposes.

The near-infrared (NIR) analyzer system will be adapted to monitor and analyze a process stream of an adsorption unit for separation of pX and EB from mX and oX. The separations are based on molecular size and comprise the selective adsorption of the smaller C₈ aromatics (pX and EB) on a non-acidic, para-selective molecular sieve such as silicalite; mX and oX pass through the bed and are not adsorbed.

Preferably, the adsorption unit will be operated as a fixed-bed, batch-wise, substantially isothermal adsorption process conducted at substantially constant total pressure. In such processes, also termed purge swing processes, desorption and regeneration is accomplished by purging or sweeping with an inert gas, thereby reducing the adsorbate partial-pressure over the adsorbent bed.

As employed herein, the term “substantially isothermal” means that the only change in temperature of the adsorbent during the process cycle is due to the heats of adsorption and desorption. The terms “substantially constant pressure” and “substantially constant operating pressure” are intended to mean that the adsorption unit remains at a constant total pressure, with minor variations in pressure due to changes in flows, etc.

The adsorption process employs a para-selective adsorbent for separation of pX and EB from mixed C₈ aromatics. By “para-selective adsorbent” is meant an adsorbent that, when subjected to an equal molar mixture of C₈ aromatics at 50° C., adsorbs pX and EB preferentially, such that the total pX and EB in the adsorbate is at least about 75%, and preferably greater than about 75% relative to the total C₈ aromatics.

The adsorbent may be further characterized as a para-selective, non-acidic, molecular sieve, preferably a medium pore molecular sieve, selected from the group of molecular sieve structure types consisting of MFI, TON, MTT, EUO, MEL, and FER.

Adsorbents which are particularly suitable include non-acidic molecular sieves of the MFI structure type such as, for example, Na—ZSM-5. These have the same structure as the acidic zeolite ZSM-5 molecular sieves but with the acid sites replaced with neutral moieties so that the molecular sieve is non-catalytic and does not isomerize xylenes. The preferred adsorbent is silicalite, an all silica, isostructural form of ZSM-5. Like H—ZSM-5, silicalite selectively adsorbs pX and EB. However, unlike H—ZSM-5, silicalite contains no acid sites, and pX and EB are thus selectively adsorbed because of their smaller molecular size. Since the molecules are only adsorbed physically, and not chemically as with H—ZSM-5, the desorption rates are high and the cycle times are shorter. Moreover, pX does not isomerize, even at the elevated temperatures necessary to make the separation process economically practicable. Use of a non-acidic molecular sieve eliminates the occurrence of undesirable catalytic reactions of the adsorbed EB and pX, and, because non-acidic silicalite is less subject to adsorption of olefin contaminants, the adsorption capacity does not decrease significantly with repeated adsorption/desorption cycles due to adsorption of olefin components in the aromatic stream.

Silicalite molecular sieve, comprising orthorhombic crystals having an average minimum dimension of around 0.2 μm or greater, has high para-xylene and ethylbenzene selectivity. During adsorption, mX and oX are substantially not adsorbed, while pX and EB are substantially adsorbed at a higher partial pressure, and selectively desorbed with no isomerization at a lower partial pressure.

The para-xylene adsorption capacity of the silicalite adsorbent is at least 1 wt %, preferably at least 2 wt %, and most preferably from about 3 to about 15 wt % at saturation. Adsorbent capacity is typically defined as grams adsorbate (i.e., material adsorbed) divided by grams adsorbent and can also be expressed as a weight percent by multiplying by 100.

Non-acidic molecular sieves of the MEL structure type are microporous materials having similar pore size and adsorption capacity to MFI molecular sieves, and, as such, would be expected to behave similarly. Both MFI and MEL molecular sieves are classified as medium pore molecular sieves. Other medium pore molecular sieves that may find use in the present invention are structure types MTW (12 ring structure, e. g., ZSM-12), ATO (12 ring structure, e. g., ALPO-31), NES (10 ring structure, e. g., Nu-87), TON (10 ring structure, e.g., Theta-1, ZSM-22), MTT (10 ring structure, e.g., ZSM-23), FER (10 ring structure), EUO (10 ring structure), MFS (10 ring structure, e.g., ZSM-57), AEL (10 ring structure, e.g., ALPO-11), AFO (10 ring structure, e.g., ALPO-41), and SUZ-4 (10 ring structure).

Large pore molecular sieves, such as mordenite, zeolite Beta, and faujasites, and amorphous adsorbents, such as silica, alumina, and clays, are non-selective, and therefore undesirable for use in the present invention, while small pore zeolites, such as zeolite A, are too small to admit pX and EB into the pores.

The adsorbent may further comprise a binder, preferably selected from the group consisting of clay, alumina, silica, titania, zirconia, silica-alumina, silica-magnesia, silica-zirconia, silica-thoria, silica-beryllia, silica-titania, silica-alumina-thoria, silica-alumina-zirconia, silica-alumina-magnesia, silica-magnesia-zirconia, and aluminum phosphate. Preferably, the adsorbent will contain from about 5 to about 100 weight percent molecular sieve.

The adsorbent may be contained in one or more containers or vessels in which separation may be effected using programmed flow into and out of the container or vessel. The vessel is described as having an inlet, or lower port, and an outlet, or upper port. Although such vessels are typically disposed vertically with a physical upper port and a physical lower port, it will be understood that by lower port or inlet is meant the port where the hydrocarbon feed stream is admitted to the vessel, and by upper port or outlet is meant the port where the unadsorbed hydrocarbon effluent stream exits the vessel during the adsorption step. During the desorption step of the cycle, the flow is preferably countercurrent to the feed. As a result of the countercurrent flow, it will be understood that by upper port or inlet is meant the port where the inert gas sweep is introduced, and by lower port or outlet is meant the port where the desorbed hydrocarbon product exits the vessel during the desorption step.

In a purge swing process, the cycle will include a charging or adsorption step and a purging or desorption step. The adsorption step will be operated by first passing a mixed hydrocarbon stream containing EB, pX, mX and oX over a fixed bed of silicalite at high pressure. The pX and EB components will be adsorbed; mX and oX components will be substantially not adsorbed and pass through the bed. A stream substantially enriched in mX and oX may thus be collected at the upper port of the bed during the adsorption step until the bed is saturated and the feed is discontinued.

After saturation of the silicalite, the desorption step will be conducted by feeding a purge gas such as CH₄, CO₂, He, H₂ or N₂ to the adsorbent bed, preferably countercurrently, while maintaining a constant total pressure. The feed contained in the non-selective void volume will be displaced by the purge gas and, in a countercurrent sweep, removed through the lower port, followed by a stream enriched in pX and EB, desorbed by the reduced EB and pX partial-pressures over the adsorbent bed as a result of the inert gas sweep. At operating temperatures greater than 176° C., preferably greater than about 230° C., the rates of both adsorption and desorption are fast, minimizing cycle time.

The cycle time is the interval of time starting when feed is admitted into a vessel and ending at the time the vessel is purged and ready for the next addition of feed. The cycle time can thus be characterized as the time interval at which feed to a pressurized adsorbent vessel is initiated, e.g., every 1 minute, every 5 minutes, every 10 minutes, every 15 minutes, etc.

The adsorption process will be carried out in the vapor phase. Conditions for the process include temperatures from about 176° C. to about 400° C., preferably from about 250° C. to about 400° C., more preferably from about 315° C. to about 370° C., and sufficient to maintain components in the vapor phase at system pressures from about 30 psia (690 kPa) to about 400 psia (2760 kPa), preferably from about 150 psia (1030 kPa) to about 350 psia (2410 kPa), more preferably from about 200 psia (1380 kPa) to about 300 psia (2070 kPa), and with pX partial pressures of from about 30 to about 150 psi (from about 200 to about 1000 kPa), preferably from about 40 to about 120 psi (from about 265 to about 800 kPa).

The components in the streams from the adsorption unit may be further separated by methods known in the art to provide substantially pure para-xylene, ethylbenzene, ortho-xylene, and meta-xylene products.

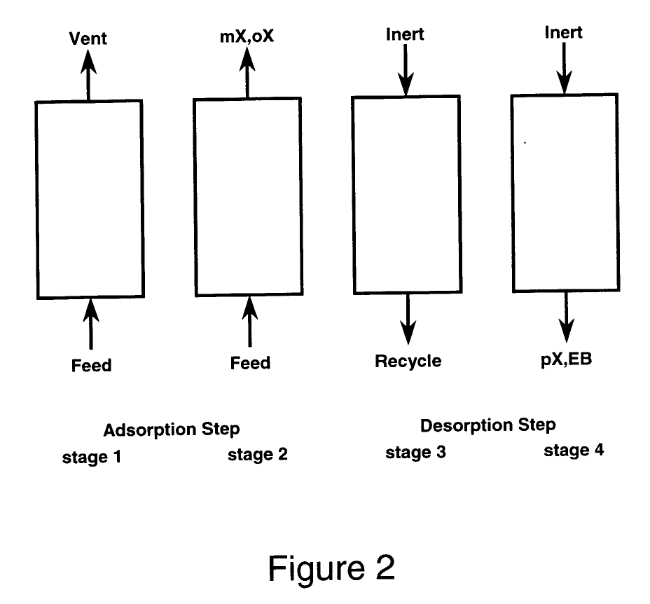

The invention will be better understood from a consideration of a preferred operating embodiment of the adsorption process, illustrated schematically in FIG. 2. Specific terminology used for the sake of clarity is not intended to be limiting; it will be understood that each specific term includes all technical equivalents which operate in a similar manner to accomplish a similar purpose.

In the descriptions that follow, the adsorbed phase, principally pX and EB, may contain other adsorbable components such as benzene, toluene, 1,4-methylethylbenzene, 1,4-diethylbenzene, linear paraffins (typically C₉) and mono-methyl branched paraffins (also typically C₉). Likewise, the non-adsorbed phase, principally mX and oX, may contain other non-adsorbable components such as trimethylbenzenes, other isomers of methylethylbenzene and diethylbenzene, cycloparaffins (typically C₉), and other sterically bulky components in the feed.

The illustrated embodiment comprises a gas-phase process wherein the temperature is substantially isothermal and the total pressure is substantially constant. The pressure and temperature are selected to allow for rapid adsorption and desorption, leading to rapid loading and unloading of the adsorbent bed. Cycle times may be as short as about 30 seconds to as great as about 30 minutes, preferably no more than about 25 minutes, more preferably no more than about 20 minutes, still more preferably about 1 to about 15 minutes, and most preferably, about 3 to about 15 minutes. Shorter cycle times reduce the amount of adsorbent required.

The adsorbent vessel will preferably comprise a fixed adsorbent bed. A suitable bed of molecular sieve adsorbent typically contains about 20-30% of its volume in molecular sieve pores which selectively adsorb pX and EB and 80-70% of void space and large non-selective pores.

The sweep or purge gas will be a vapor or gas over the range of temperatures and pressures employed in the process, and will be inert with respect to the hydrocarbon components and selected to be unreactive with respect to the adsorbent. Gases which may be suitable for these purposes include CH₄, CO₂, H₂, N₂, and He.

The purge swing process is described in greater detail in U.S. patent application Ser. No. 09/902,198 as a pressure swing adsorption with inert purge gas.

Turning now to FIG. 2, describing the operating cycle of the adsorption column, the purge swing process will be seen to comprise two phases or steps, an adsorption step and a desorption step, each including two stages. The adsorbent column or vessel will be purged prior to admitting C₈ aromatic feed and thus will contain sweep gas at the operating pressure and be substantially free of hydrocarbons, including C₈ aromatics, at the start of the cycle. One complete cycle is described; it will be understood that the practice of the process involves principally proceeding by repeating said cycles.

Stage 1: Displacement of Purge Gas and Initial Adsorption of pX and EB

Feed comprising a mixture of substantially C₈ aromatics (mX, oX, pX, EB) is passed through the lower port and into the adsorption vessel, displacing the purge gas. The pX and EB components are adsorbed into the pores of the molecular sieve, leaving mX and oX in the void space. As the front of the feed flow advances, the purge gas is displaced through the vessel toward the upper port of the vessel, passing through the upper sample cell (if so equipped). Stage 1 is complete when the purge gas is substantially displaced from the void fraction.

Stage 2: Production of mX and oX and Saturation of the Adsorbent with pX and EB

As the feed continues to enter the adsorption vessel, pX and EB continue to adsorb into the molecular sieve and mX and oX are displaced from the void fraction by incoming feed. The unadsorbed components, mX and oX, exit the vessel at the upper port and pass through the sample cell as an effluent stream, which may be substantially free of pX and EB. The feed will be discontinued and collection of the mX and oX effluent stream stopped just prior to breakthrough of the feed at the upper port of the vessel.

At this point the vessel will contain adsorbed pX and EB in the molecular sieve, together with feed mixture in the void fraction.

Stage 3: Displacement of the Feed from the Void Fraction

Sweep or purge gas is fed to the adsorption vessel, typically countercurrent to the flow of C₈ aromatics during the feed step.

The pX and EB are more strongly adsorbed inside the pores of the molecular sieve than the feed mixture in the void fraction. As purge gas enters the reactor at the upper port, the feed mixture contained in the void fraction will thus be displaced through the lower port and pass through the lower sample cell (if so equipped). This portion of the effluent, substantially of feed composition, typically would be recycled back to the feed stream for the adsorption process.

Stage 3 is complete when essentially all of the feed mixture, possibly together with minor amounts of desorbed pX and EB, has been purged from the vessel, i.e. when desorbed pX and EB components of the purge stream reaching the lower port are substantially increased.

Stage 4: Production of pX and EB

Once the feed is displaced from the void fraction, the effluent becomes highly enriched in pX and EB. Since the purge gas lowers the partial pressure of pX and EB in the adsorbent vessel, pX and EB continue to desorb from the molecular sieve and are swept from the adsorbent vessel through the lower port and sample cell.

At the end of stage 4, when the purge gas stream reaching the lower port and the sample cell contains minimal hydrocarbon component, the void fraction and molecular sieve pores are essentially filled with purge gas, and the system is in a condition to begin Stage 1 again.

It will be apparent that determining changes in the composition of the process streams passing through the upper and lower sample cells will provide information needed to control and optimize the cycle timing. Thus, monitoring the absorbance of the effluent during the adsorption step will detect the presence of hydrocarbon at the end of stage 1 and signal the timing of the collection of the mX/oX stream, while analysis of the major component levels will identify the increase in the relative level of pX due to breakthrough at the end of stage 2, signaling the timing for ending the feed and beginning the inert gas sweep. Similarly, monitoring the effluent during the desorption step to determine change in the major component levels will detect the increase in the relative levels of pX and EB at the end of stage 3 and the reduction in the hydrocarbon level at the end of stage 4, signaling the timing for the collection of the pX/EB stream. Timing these events in real-time, rather than through empirically derived process models, affords a more precise timing and improved process efficiency.

A detailed interpretation of the changes in the composition of the process steams during the adsorption and desorption steps will also provide information about the kinetics of these processes. Thus, the real-time monitoring will provide information to control and optimize the feed and purge gas flow rates and the operating temperatures and pressures of the adsorption vessels.

The invention will be better understood by consideration of the following examples which are provided to illustrate certain embodiments.

### EXAMPLES

A pilot scale separation apparatus comprising two adsorption columns as generally described and shown in FIG. 2, charged with Silicalite and operated substantially as described, is fitted with an NIR analyzer system including components as follows:

Fourier-transform near-infrared (FT-NIR) spectrometer, obtained from Hamilton Sundstrand, Pomona, Calif.

fiber optic multiplexer, obtained from Axiom Analytical, Irving, Calif.

sample cell having a path length of 10 cm, obtained from Axiom Analytical, Irving, Calif.,

optical attenuator, obtained from Axiom Analytical, Irving, Calif.

Indium-Gallium-Arsenide (InGaAs) detector, obtained from Galileo Electro-Optics, Sturbridge, Mass.

Optical connections between the FT-NIR, multiplexer, sample cell, attenuator and InGaAs detector were made using 300-micron diameter core, low-OH silica fiber optic cables about 1-meter long, obtained from Polymicro Technologies, Phoenix, Ariz. The entire NIR analyzer system is under computer control using the FT-NIR process monitoring software PC80, provided by Hamilton Sundstrand, Pomona, Calif., real-time chemometric prediction software “InStep” from Infometrix, Seattle, Wash., and several custom software programs.

Reference or background spectra were obtained by setting the multiplexer to the attenuator channel. Sample, or foreground spectra were measured by the NIR analyzer system by setting the multiplexer to the sample cell channel. The attenuator is adjusted to vary the light throughput so that the signal level is approximately the same as the sample cell channels. This allows for the same amplifier gain to be used on all channels and to optimize the signal-to-noise ratio of the spectrum. The negative base-10 logarithm for the ratio of the foreground over the background spectrum is the absorbance spectrum of the sample.

The upper ports of the adsorption columns are manifolded and valved such that at least a portion of the effluent stream during the adsorption step from both columns may be directed into and passed through the sample cell. Likewise, the lower ports of the adsorption columns are manifolded and valved such that at least a portion of the effluent stream during the desorption step from both columns may be directed into and passed through the sample cell.

### Example 1

A mixture comprising oX (22.4 wt %), mX (49.8 wt %), pX (22.4 wt %), and EB (5.4 wt %) is fed to the adsorption apparatus, maintained at a temperature of 220° C., operated at substantially constant pressure of 60 psia, with a hydrocarbon feed rate of about 15 grams/minute, and operated using a nitrogen sweep gas flow rate of 10 standard-cubic-feet-per-hour.

The sample cell, fitted to the manifolded upper ports of the two columns, monitors the hydrocarbon content of the mX/oX stream from the adsorption process. The feed stream is initiated to the lower port of the first said column, maintained for a period sufficient to charge the adsorbent bed, then switched to the second said column and continued for a like period, then re-directed to the first column. The nitrogen sweep gas is fed countercurrently to the first column to accomplish the desorption step while the second column is being charged, and to the second column while the first column is again charged. The process continued for a period of about 4.5 hr, using operating cycles (adsorption plus desorption) of approximately 8 min.

The hydrocarbon stream passing through the sample cell from the upper ports is monitored by NIR analysis system. The absorbance values are measured at wavenumbers of 6495.0, 5978.2, 5908.8, 5893.4, 5847.1 and 5785.4 cm⁻¹, respectively.

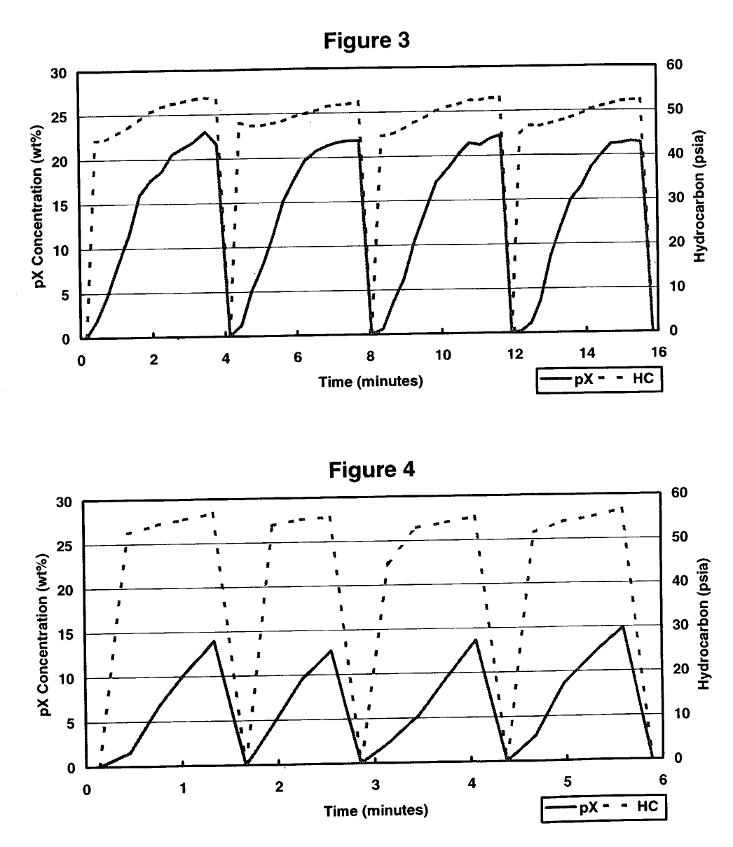

The data for two such cycles (two adsorption steps for each of the two columns) are shown graphically in FIG. 3. It will be seen that the hydrocarbon partial pressure in the effluent increases very rapidly with time and levels off at a fairly constant value once the purge gas in the void space of the column has been displaced. The concentration of pX in the hydrocarbon phase similarly increases,, but at a slower rate. The concentration increases from zero up to about 22 wt %, which is approximately the pX concentration of the feed, and essentially levels off for close to 30% of the feed cycle.

In this run the operating conditions (cycle timing, flow rates, temperatures, and pressures) are arbitrary; it will be apparent that the detection method provides a signal dependent upon the hydrocarbon partial pressure and the pX content of the effluent that may be useful to control and optimize these operating conditions.

### Example 2

The adsorption apparatus was again operated substantially as described in Example 1, but using substantially shorter cycle times. The hydrocarbon content of the mX/oX stream from the adsorption process was repeatedly determined over a period of about 1.8 hr., using operating cycles (adsorption plus desorption) of approximately 3 min.

The data for two such cycles (two adsorption steps for each of the two columns) are shown graphically in FIG. 4. It will be seen once again that the hydrocarbon partial pressure in the effluent increases very rapidly with time and levels off at a fairly constant value once the purge gas in the void space of the column has been displaced.

The concentration of pX in the hydrocarbon phase still increases during the adsorption stage, but reaches a much lower value than in Example 1 when the feed is switched to the other column. The pX concentration spans from zero to about 10 wt %, reflecting a reduction in the amount of pX in the mX/oX effluent stream, and a significant improvement in the pX recovery, as compared to Example 1, by decreasing the cycle time.

The immediate breakthrough of small amounts of pX from the beginning of the adsorption step and the steady increase in Examples 1 and 2 suggest that the feed rate exceeds the rate of adsorption under the temperature and pressures conditions of these runs. Thus, the process conditions can still be further optimized using the real-time compositional data.

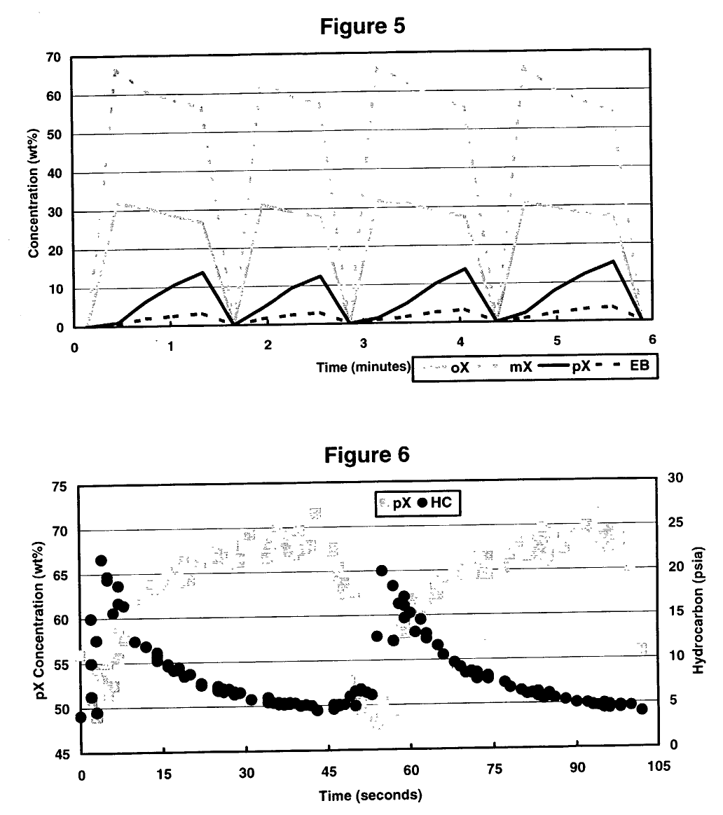

The concentration of the meta-, ortho- and para-xylene, and ethylbenzene components in the hydrocarbon phase of the mX/oX effluent stream for the two cycles, estimated using the PLS models, are shown graphically in FIG. 5. It will be seen that the time dependent behavior of the mX and oX concentrations are quite similar because they are both substantially not adsorbed in the columns. The time dependent behavior of the pX and EB concentrations are also quite similar, and opposite that for mX and oX, because they are both substantially adsorbed in the columns.

### Example 3

The adsorption apparatus is again operated substantially as described in Example 1, except the feed rate is about 20 grams/minute. In this run the sample cell is fitted to the manifolded lower ports of the two columns to monitor the hydrocarbon content of the pX/EB stream being swept from the adsorption columns with nitrogen during the second part of the desorption step, or stage 4. The contents of the void volume of the adsorption columns, which is swept from the adsorption columns during stage 3 is diverted prior to sample cell so that only the pX/EB rich material desorbed from the adsorbent will be measured.

The process continued for a period of about 26 min., using operating cycles (adsorption plus desorption) of approximately 100 sec. The hydrocarbon content is repeatedly determined using baseline and hydrocarbon absorbances measured at wavenumbers of 6495.0 and 5893.4 cm⁻¹, respectively. The pX concentration is repeatedly estimated using the additional absorbances measured at wavenumbers of 5978.2, 5908.8, 5847.1 and 5785.4 cm⁻¹.

FIG. 6 presents the data as overlaid profiles of the dual column sequence for about 14 complete cycles. The left half of the plot corresponds to the stage 4 sweep of column 1 and the right half is column 2.

At the beginning of stage 4, the hydrocarbon partial pressure in each of the columns has been reduced to about 20 psia, from the operating pressure of 60 psia, due to removal of the feed in the void volume of the adsorption column. The effluent stream contains the pX/EB being desorbed from the column that is diluted with nitrogen sweep gas. As stage 4 continues, the hydrocarbon partial pressure of the effluent continues to decrease as pX/EB desorption is completed.

The concentration of pX in the hydrocarbon phase is approximately 50 wt % at the beginning of stage 4, which is significantly higher than the feed concentration of 22.4 wt %, and represents the final portions of the feed in the void volume and the start of the pX and EB that are being desorbed from the adsorbent. As stage 4 continues, the pX concentration continues to increase to nearly 70 wt %.

It will be apparent that the decrease in hydrocarbon partial pressure and increase in pX concentration serve to provide reproducible real-time signals that may be used to control and optimize of the cycle timing and sweep gas flow rate. It will be further apparent that the concentration of pX in the pX/EB rich stream recovered during stage 4 serves to produce a reproducible real-time signal that may to used to control and optimize additional operating conditions.

### Example 4

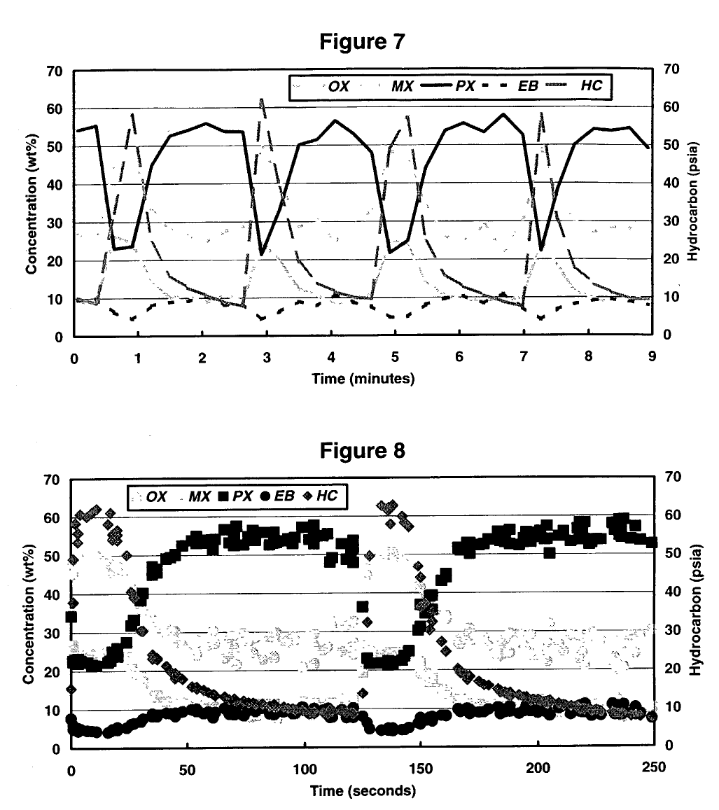

The adsorption apparatus is again operated substantially as described in Example 1, as described in Example 1, except the feed rate is about 9 grams/minute and the sweep gas flow rate is 6.75 standard-cubic-feet-per-hour. The sample cell is fitted to the manifolded lower ports of the two columns to monitor the hydrocarbon content of the process stream being swept from the adsorption columns during the entire desorption step (stage 3 and stage 4). Spectra of the effluent stream from the adsorption apparatus are repeatedly measured in real-time over a period of about 45 min., using operating cycles (adsorption plus desorption) of approximately 4 min. The hydrocarbon partial pressure and the concentration of ortho-, meta- and para-xylene, and ethylbenzene are determined in real-time using PLS models. The data are presented graphically in FIGS. 7 and 8

FIG. 7 presents the continuous trend plot for 2 complete cycles of each of the two columns. The HC trace is the partial pressure of hydrocarbon and the oX, mX, pX, and EB traces are the percent concentration of each component in the hydrocarbon phase. During stage 3, the initial part of the sweep, the residual feed is being swept from the void volume. When the sweep begins, the hydrocarbon partial pressure is about 59 psia, which corresponds quite well with the operating pressure of the adsorption columns, and the oX, mX, pX, and EB concentrations are about 25 wt %, 48 wt %, 22 wt %, and 5 wt %, respectively, which correspond quite well with the feed composition.

After residual feed has been mostly flushed from the void volume, the oX and mX concentrations will be seen to decrease quickly, and the pX and EB concentrations are seen to rapidly increase, signaling the beginning of stage 4. The hydrocarbon partial pressure simultaneously decays in an exponential fashion as the stream increases in nitrogen concentration. At the end of stage 4, the HC content is about 10 psia, and the C₈ aromatic concentrations are about 10 wt %, 25 wt %, 55 wt %, and 10 wt % for oX, mX, pX, and EB, respectively, or about 65 wt % combined pX and EB.

FIG. 8 presents the overlaid profiles of the dual column sequence for about 10 complete cycles of the desorption. The first half of the plot corresponds to the sweep of column 1 and the second half is column 2. The exponential decay of the hydrocarbon partial pressure, the decrease in oX and mX content, and the increase in pX and EB as the desorption step proceeds will be apparent.

With the particular combination of instrumentation and microprocessor used in these examples, and the brief cycle times typical of such processes, only from 9 to 26 data points will be obtained and calculated per cycle. Faster spectrometers and microprocessors are available, and it will be recognized that such instrumentation may be usefully employed where more frequent results are desired.

It will be apparent that analysis of the mX/oX stream using the NIR analyzer thus shows when the pX and EB breakthrough occurs for the adsorbent column where they have been preferentially adsorbed. This indicates that the absorbent in the column has been saturated or is being fed at too fast of a rate. Data from the NIR analyzer thus allows the operator to control and optimize the feed rate and overall timing of the adsorption step (stage 1 and stage 2), assuring a high recovery of pX (and EB).

Similarly, analysis of the pX/EB stream using the NIR analyzer shows the concentration profiles and the total hydrocarbon, allowing the operator to control and optimize the sweep gas flow rate and the cycle timing between diverting the approximately feed composition material swept from the void volume of the adsorbent column (stage 3) and collecting the pX/EB rich effluent that is a desired product (stage 4). This controls a trade off between the pX/EB product purity and the amount of diverted hydrocarbon material. The diverted hydrocarbon material can be recycled and combined with the feed, thus these conditions influence the amount of fresh feed that can be charged to the adsorption unit.

The operator may also use the information from the analyzed streams to control other process parameters including temperature and pressure of the process to optimize the total recovery and purity of the product streams. These analyses also provide information about the selectivity and capacity of the adsorbent.

The NIR analyzer may also be used on the feed stream to perform feed forward control of the adsorption process. The feed will generally be a process stream having compositional variability, and possibly the addition of a recycle stream (from stage 3) to the fresh feed to create the total feed. Knowledge of the feed composition is useful in controlling many of the process parameters.

Data provided by the analyzer may be output in readable form for use in manual operation. More preferably, for microprocessor-controlled processes the output of the analyzer will be supplied as a signal to a microprocessor programmed to control the process parameters.

It will be understood that alternative methods for operation of the adsorption process are also possible. For example, the cycle may include pressure swing adsorption (PSA) stages in which the absolute pressure within the adsorbent vessel is varied; the absolute pressure is reduced to desorb the pX/EB component, and then raised to enhance adsorption of the pX/EB component from the feed stream. In a further embodiment a rinse stream of substantially pure pX/EB is used to displace feed from the non-selective void volume prior to lowering the absolute pressure within the adsorbent vessel for desorption of pX/EB. In yet another embodiment, pX/EB is desorbed by lowering the absolute pressure within the adsorbent vessel, and displaced from the non-selective void volume by a purge stream of substantially pure mX/oX. The process may be operated whereby depressurization occurs in at least two steps, such that gas from depressurization is used to pressurize a regenerated bed (i.e., the cycle contains at least one pressure equalization step). In a further modification, a PSA cycle employing pressure equalization, a pX/EB rinse step prior to desorption of pX/EB by depressurization, and an mX/oX purge step may be employed. The preferred constant-pressure-with-inert-gas-purge operation of the process, together with alternative methods of operation, are set forth and described in greater detail in the aforesaid U.S. patent application Ser. No. 09/902,198.

While the invention has been illustrated by means of specific embodiments, these are not intended to be limiting. Further additions and modifications will be readily apparent to those skilled in the art, and such modifications and additions, and processes and apparatus embodying them, are contemplated to lie within the scope of the invention as defined and set forth in the following claims.

1. In a para-xylene production unit comprising a catalyst reactor for isomerization of aromatics, a catalyst reactor for ethylbenzene conversion, and one or more distillation columns for separation of aromatics, an adsorption unit for separation of components of a C₈ aromatic hydrocarbon stream and, optionally, a fractional crystallization unit for separating para-xylene from ethylbenzene, the improvement wherein said unit further comprises a near-infrared analyzer system adapted for determining the presence of at least one hydrocarbon component in a stream thereof.

2. The para-xylene production unit of claim 1 wherein said hydrocarbon component is selected from the group consisting of total hydrocarbon, para-xylene, ethyl benzene, ortho-xylene, and meta-xylene.

3. The para-xylene production unit of claim 1 wherein said hydrocarbon component is determined by a process including the steps of:
(a) advancing a stream or a portion thereof through a sample cell fitted with a near-infrared source to emit near-infrared radiation and adapted to pass the near-infrared radiation through the effluent stream and a detector to detect transmitted near-infrared radiation;
(b) measuring the portion in the sample cell with NIR energy at a plurality of NIR wavelengths, wherein each of the components absorbs NIR energy at one or more of the plurality of NIR wavelengths;
(c) detecting the NIR energy passing through the sample cell and generating absorbance data for each of the components; and
(d) quantifying each of the components by applying the absorbance data to a calibration equation for each component in a microprocessor programmed to quantify each of the components.

4. The para-xylene production unit of claim 3 wherein said microprocessor outputs a signal or signals indicative of one or more hydrocarbon components to control an operation of a para-xylene production process.

5. The para-xylene production unit of claim 3 wherein said microprocessor outputs the quantified data in readable form.

6. The para-xylene production unit of claim 1 wherein said stream of said C₈ aromatic hydrocarbons comprises para-xylene, ethylbenzene, ortho-xylene, and meta-xylene.

7. The para-xylene production unit of claim 1 wherein near-infrared (NIR) analyzer system includes a microprocessor programmed to quantify each of the components of a stream.

8. The para-xylene production unit of claim 7 wherein said microprocessor outputs a signal or signals indicative of one or more hydrocarbon components to control an operation of said para-xylene production unit.

9. The para-xylene production unit of claim 1 wherein para-xylene selective adsorption means comprises an adsorbent vessel containing a para-selective, non-acidic, medium pore molecular sieve.

10. An apparatus for recovery of para-xylene from a C₈ aromatic hydrocarbon stream comprising a near-infrared (NIR) analyzer system and an absorption unit comprising an adsorbent vessel containing a fixed adsorbent bed, said NIR analyzer system adapted to determining the presence of at least one hydrocarbon component in a stream of said absorption unit.

11. The apparatus of claim 10 wherein said adsorbent bed comprises a para-selective, non-acidic, medium pore molecular sieve.

12. The apparatus of claim 11 wherein said para-selective, non-acidic, medium pore molecular sieve is selected from the group of molecular sieve structure types consisting of MFI, TON, MTT, EUO, MEL, and FER.

13. The apparatus of claim 11 wherein said para-selective, non-acidic, medium pore molecular sieve is silicalite comprising orthorhombic crystals having an average minimum dimension of about 0.2 μm.

14. The apparatus of claim 11 wherein said para-selective, non-acidic, medium pore molecular sieve contains about 20-30% of its volume in molecular sieve pores which selectively adsorb pX and EB and 80-70% of void space and large non-selective pores.

15. The apparatus of claim 10 wherein said hydrocarbon component is selected from the group consisting of total hydrocarbon, para-xylene, ethylbenzene, ortho-xylene, and meta-xylene.

16. The apparatus of claim 10 wherein said near-infrared analyzer system is adapted for quantitatively determining the total hydrocarbon, ethylbenzene, para-xylene, meta-xylene and ortho-xylene components of a stream thereof.

17. The apparatus of claim 10 wherein said near-infrared analyzer system outputs a signal or signals indicative of one or more hydrocarbon components for controlling an operation of a para-xylene recovery process.

18. A para-xylene production unit comprising catalytic means for isomerization of aromatics, distilling means for separation of C₈ aromatic hydrocarbons from a hydrocarbon stream from said catalytic means, para-xylene selective adsorption means for separation of para-xylene from a stream of said C₈ aromatic hydrocarbons from said distilling means, thereby providing a stream enriched in para-xylene and a stream reduced in para-xylene, relative to the para-xylene content of said stream of said C₈ aromatic hydrocarbons, and near-infrared spectroscopic means for determining the presence of at least one hydrocarbon component of one or more of said hydrocarbon streams.

19. The para-xylene production unit of claim 18 further comprising fractional crystallization means for separating para-xylene from a hydrocarbon stream comprising para-xylene and ethylbenzene.

20. The para-xylene production unit of claim 18 further comprising catalytic means for ethylbenzene conversion.
